(19) United States
(12) Patent Application Publication
(10) Pub. No.: US 2014/0012270 A1
(43) Pub. Date: Jan. 9, 2014

(54) JOINT STABILIZING INSTRUMENT AND METHOD OF USE

(71) Applicant: STRYKER CORPORATION, Kalamazoo, MI (US)

(72) Inventors: John R. Fossez, Frisco, TX (US); Stuart L. Axelson, JR., Succasunna, NJ (US); Timothy J. LaRoy, Kalamazoo, MI (US); Dave J. Veldkamp, Grand Rapids, MI (US); Dustin L. Worm, Plainwell, MI (US)

(73) Assignee: STRYKER CORPORATION, Kalamazoo, MI (US)

(21) Appl. No.: 13/790,611

(22) Filed: Mar. 8, 2013

Related U.S. Application Data
(60) Provisional application No. 61/667,583, filed on Jul. 3, 2012.

(51) Int. Cl. A61B 17/02

(52) U.S. Cl. CPC A61B 17/025
USPC 606/90

(57) ABSTRACT

A joint spacer having a body and a series of extensions projecting therefrom is disclosed. A first of the series of extensions may include a curved section for housing a portion of a joint therein (e.g., the intercondylar notch of a knee), and a second and third of the extensions may be configured to abut an opposing portion of the joint. Once inserted, the joint spacer may maintain the spacing between, and the stabilization of, the joint during surgery. A fourth extension may be included with the spacer in which the extension may interact with an insertion-removal instrument. The insertion-removal instrument may include one end having an insertion geometry for use in inserting the spacer into the joint, and an opposing end having a removal geometry for removing the spacer. Related methods for inserting the joint spacer and various alternate joint distraction devices are also disclosed.

### CROSS-REFERENCE TO RELATED APPLICATIONS

The present application claims the benefit of the filing date of U.S. Provisional Patent Application No. 61/667,583, filed Jul. 3, 2012, the disclosure of which is hereby incorporated by reference herein.

### BACKGROUND OF THE INVENTION

The present invention(s) relates generally to various spacers and/or distraction instruments, and more particularly to spacers or distraction instruments configured to maintain the spacing between contiguous bone segments during surgery (e.g., knee surgery).

In traditional knee arthroplasty surgery, the diseased bone and/or cartilage of a patient is typically removed and replaced with a prosthetic implant. An example of a prosthetic implant for use in an arthroplasty surgery is set forth in U.S. patent application Ser. No. 13/530,927 (the “'927 Application”), the disclosure of which is hereby incorporated by reference herein in its entirety.

To accommodate a prosthetic implant, of the type disclosed in the '927 Application or otherwise, a surgeon typically prepares a patient's bone, in some cases the proximal tibia and the distal femur, using a hand-held oscillating saw blade or other cutting instrument (e.g., planar resection guides, drills, chisels, punches, reamers, rotational burrs, or the like). Specifically, the surgeon may conduct a series of resections, which may result in the formation of a series of planar bone surfaces on the diseased bone to be treated. Additionally, in some cases, the surgeon may use a drill, broach, or tamp instrument to form cylindrical holes into the resections formed in the bone site to accommodate peg fixation features, which may be included on the prosthetic implant. The planar bone resections and cylindrical bone holes may be oriented to interface, respectively, with the flat bone contacting surfaces and pegs of the prosthetic implant.

Unicondylar knee replacement (hereinafter “UKR”) is an exemplary procedure where the distal portion of one condyle of the femoral bone and the corresponding proximal tibial bone may be prepared via the cutting instrumentation noted above. During such preparation of bone, it is critical that the knee joint remains stable and that the relative distance between the distal femoral and proximal tibial bones is maintained. When this space is not maintained, it is possible for the bones (e.g., the tibia and the femur) to essentially collapse on the cutting tool used, which can cause a less than optimal result in bone preparation and have negative consequences for the patient.

A challenge with maintaining joint stability and relative distance between the distal femur and the proximal tibia is that the cutting instrumentation used may require a certain working volume around an opened joint capsule. Therefore, use of a standard retraction instrument (e.g., a Gelpi retractor) for stabilizing the joint may cause interference with the working area of the cutting tool being utilized (e.g., the handles of the Gelpi or other retractor may interfere with the working area of the cutting tool used to prepare bone). Such retraction instruments may also cause damage to anatomy not planned for resection. Simply stated, depending on the configuration of the retraction instrument, the cutting instrumentation used may bump into the handle of the retraction tool(s), thus interrupting bone preparation during knee arthroplasty, and possibly causing damage to the surrounding anatomical structures.

While there are devices that maintain the spacing between the distal femur and proximal tibia during knee arthroplasty procedures, as described, such devices, and their corresponding uses, may be expanded and improved upon.

### BRIEF SUMMARY OF THE INVENTION

A first aspect of the present invention provides a joint spacer comprising a body having first, second, and third extensions projecting therefrom, the second and third extensions being spaced apart from one another, with each extension having a bone contacting surface. Further, the first aspect contemplates that the first extension may lie in a plane extending between the spaced apart second and third extensions and vertically above such extensions, with the first extension including a curved portion.

Embodiments of the aforementioned first aspect may also include a curved portion on the first extension, such curved portion containing a first curved section and a second curved section, the first section curving in an opposite direction to the second section. Also, the second curved section may be configured to receive a portion of an intercondylar notch of a femur, and a flat portion between the first curved section and the second curved section may be configured to prevent over insertion of the spacer past the intercondylar notch.

A second aspect of the present invention includes a joint stabilization system, which comprises a joint spacer including a body having first, second, and third extensions projecting therefrom, the second and third extensions being spaced apart from one another, and the first extension lying in a plane extending between the spaced apart second and third extensions. The system may also include a combination insertion-removal instrument having an insertion member for inserting the spacer between portions of contiguous bone segments, and a removal member for removing the spacer from between the portions of contiguous bone segments, the insertion member and the removal member being connected together through an elongate handle.

Other embodiments of this second aspect may include a joint spacer comprising a fourth extension projecting from the body of the spacer in a direction opposite to the first, second, and third extensions, the fourth extension including a bore extending at least partially therethrough. Further, the insertion member may be insertable within a portion of the fourth extension of the spacer in some embodiments.

A third aspect of the present invention contemplates a method for stabilizing a knee joint, the method comprising the steps of inserting a spacer having a body between the knee joint, such that a first extension projecting from the body is disposed between the anterior and posterior cruciate ligaments, and second and third extensions projecting from the body are disposed on adjacent sides of such ligaments. In some embodiments, during the method a portion of the first extension may engage the intercondylar notch of a knee or a portion of the knee adjacent such intercondylar notch to prevent over insertion of the spacer. The method according to this third aspect may also include a step of engaging a portion of an insertion-removal tool with a fourth extension projecting from the spacer, the fourth extension being oriented in a direction opposite to the first, second, and third extensions.

### BRIEF DESCRIPTION OF THE DRAWINGS

A more complete appreciation of the subject matter of the present invention(s) and of the various advantages thereof can be realized by reference to the following detailed description in which reference is made to the accompanying drawings in which:

FIG. 1 is a perspective view of a joint spacer, in accordance with one embodiment of the present invention.

FIG. 2 is a side view of the joint spacer of FIG. 1.

FIG. 3 is an alternate perspective view of the joint spacer of FIG. 1.

FIG. 4 is a bottom view of the joint spacer of FIG. 1, in which the distal surface of the spacer is shown.

FIG. 5 is a top view of a plurality of joint spacers, in accordance with other embodiments of the present invention, with each successive spacer overlying the other.

FIG. 6 is a side view of the plurality of joint spacers of FIG. 5.

FIG. 7 is a perspective view of an insertion and removal (hereinafter “IR”) instrument, in accordance with one embodiment of the present invention.

FIG. 8 is a perspective view of the joint spacer and IR instrument of FIGS. 1 and 7, respectively, in which such devices are oriented for placement of the spacer within a surgical site.

FIG. 9 is a perspective view of the joint spacer and IR instrument of FIGS. 1 and 7, respectively, in which such devices are oriented for removal of the spacer from a surgical site.

FIG. 10 is a perspective view of the joint spacer of FIG. 1 being placed into a knee joint.

FIG. 11 is a perspective view of the joint spacer of FIG. 1 being removed from a knee joint.

FIG. 12 is a perspective view of a joint spacer, in accordance with another embodiment of the present invention.

FIG. 13 is a perspective view of a joint spacer, in accordance with still another embodiment of the present invention.

FIGS. 14A-E are various views of a joint distractor, in accordance with an alternate embodiment of the present invention.

FIG. 15 is a close-up view of the distraction mechanism of the joint distractor of FIGS. 14A-E.

FIGS. 16A-E are various views of another joint distractor utilizing a buttress mechanism, in accordance with yet an additional embodiment of the present invention.

FIGS. 17A-B is a perspective view of a portion of the joint distractor(s) of FIGS. 14A-E and 16A-E being inserted within a knee joint.

FIGS. 18A-B are perspective views of a further variant of a joint distractor, in accordance with other embodiments of the present invention, with the distractor shown as being inserted within a knee joint in FIG. 18B.

FIGS. 19A-B are perspective views of still another distractor according to the present invention.

FIGS. 20A-B are perspective views of a distraction mechanism utilizing inflatable technology, according to yet another alternate embodiment of the present invention.

FIGS. 21A-D depict a method of distracting a joint space utilizing a shim or spacer.

### DETAILED DESCRIPTION

In describing the preferred embodiments of the invention(s) illustrated and to be described with respect to the drawings, specific terminology will be used for the sake of clarity. However, the invention(s) is not intended to be limited to any specific terms used herein, and it is to be understood that each specific term includes all technical equivalents, which operate in a similar manner to accomplish a similar purpose.

As used herein, the term “distal” means relatively farther from the heart and the term “proximal” means relatively closer to the heart; the term “inferior” means toward the feet and the term “superior” means towards the head; the term “anterior” means towards the forward facing part of the body (e.g., the face) and the term “posterior” means towards the back of the body; and the term “medial” means toward the midline of the body while the term “lateral” means away from the midline of the body. These terms are anatomical terms used mainly to describe the orientation and use of the present invention(s). However, such terms are not intended to be limiting in any way, as embodiments of the present invention(s) may be placed in a variety of orientations and within many different anatomical joints.

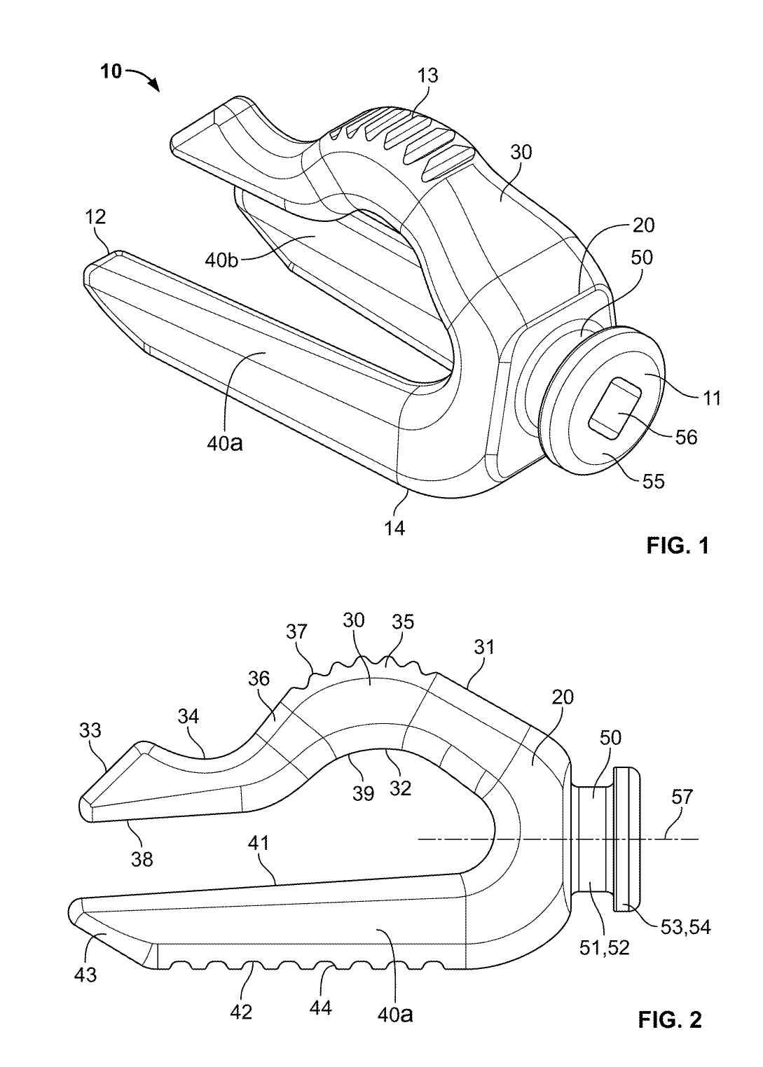

Referring to FIG. 1, a joint spacer 10 may include an anterior end 11, a posterior end 12, a proximal end 13, and a distal end 14. Further, joint spacer 10 may have a body 20 from which a series of extensions may project posteriorly, namely, a proximal extension 30 and distal extensions 40a, 40b. As will be described in detail below, spacer 10 may be used in various orthopedic procedures, such as in different knee arthroplasty surgeries, and may function to maintain joint spacing and stability during these surgeries (e.g., during bone/tissue preparation). Briefly, during a traditional knee replacement surgery, for example, spacer 10 may be inserted between the distal femur and the proximal tibia, and about the anterior cruciate ligament (hereinafter the “ACL”) and the posterior cruciate ligament (hereinafter the “PCL”), to maintain the spacing between the distal femur and proximal tibia and shield the ACL and PCL from instruments used to prepare bone during the surgery.

Referring now to FIGS. 1-2, body 20 of joint spacer may include a proximal extension 30, which incorporates a bone/tissue contacting proximal surface 31, a distal surface 32, and a lead-in surface 33. Proximal extension 30 may project generally outward from body 20 (i.e., from anterior end 11 towards posterior end 12) and terminate in lead-in surface 33. Moving in a direction from anterior end 11 to posterior end 12, surface 31 of proximal extension 30 may: (1) ascend to reach an apex (e.g., proximal end 13 of spacer 10), (2) thereafter descend to a low point; and (3) ascend once more and terminate at lead-in surface 33. Thus, proximal extension 30 may be “S-shaped” in one embodiment. Stated another way, proximal extension 30 may include a cradle region 34, an arch region 35, and a flat region 36 interposed therebetween, such regions generally forming an “S-shape.”

Distal surface 32 of proximal extension 30 may be substantially flat 38 under lead-in surface 33 and cradle region 34. Further, distal surface 32 may have a curved portion 39 opposite arch region 35. In one embodiment, the radius of curvature of curved region 39 may be substantially equivalent to that of arch region 35. Likewise, arch 35 and cradle 34 regions of proximal extension 30 may also have curved portions. Such curved portions may, in one embodiment, have radii of curvature, which may be different from one another. For instance, the radius of curvature of arch region 35 may be greater than that of cradle region 34, thus creating a gentler curvature in arch region 35 than in cradle region 34. As an example, the radius of curvature of arch region 35 may be 0.35 inches while the radius of curvature of cradle region 34 may be 0.2 inches.

Arch region 35 of proximal extension 30 may further include a series of ridges 37 thereon, which may define a sinusoidal wave pattern. Such construction may aid in maintaining spacer 10 in a desired position. Of course, other structures than those shown may be employed, including knurled surfaces and surfaces including teeth. In this embodiment, ridges 37 may be formed into arch region 35 via a milling process, through molding, or through another suitable procedure. Lead-in surface 33 may be arranged posterior to such ridges 37, and may, in one embodiment, have a tapered or angled surface. Stated differently, a distance between proximal 31 and distal 32 surfaces of proximal extension 30 may decrease at lead-in surface 33, such that proximal 31 and distal 32 surfaces form a point, which terminates proximal extension 30.

Referring still to FIGS. 1-2, spacer 10 may also include a set of distal extensions 40a, 40b, which are, in the embodiment shown in those figures, identical in geometry. Such extensions 40a, 40b are thusly described jointly below, but it is to be understood that in other embodiments the extensions may be of different geometry. As shown in FIG. 2, distal extensions 40a, 40b may have a proximal surface 41, a distal surface 42, and a lead-in surface 43. Proximal surface 41 may be substantially flat in geometry and may have a non-parallel relationship with distal surface 42. Thus, distal extensions 40a, 40b may taper in a direction extending from anterior end 11 to posterior end 12. Further, lead-in surface 43 of distal extensions 40a, 40b may be tapered (e.g., the distance between proximal surface 41 and distal surface 42 may decrease at lead-in surface 43) much like lead-in surface 33 of proximal extension 30. In another embodiment, distal surface 42 of extensions 40a, 40b may be angled or curved upwards to cradle tibial eminence.

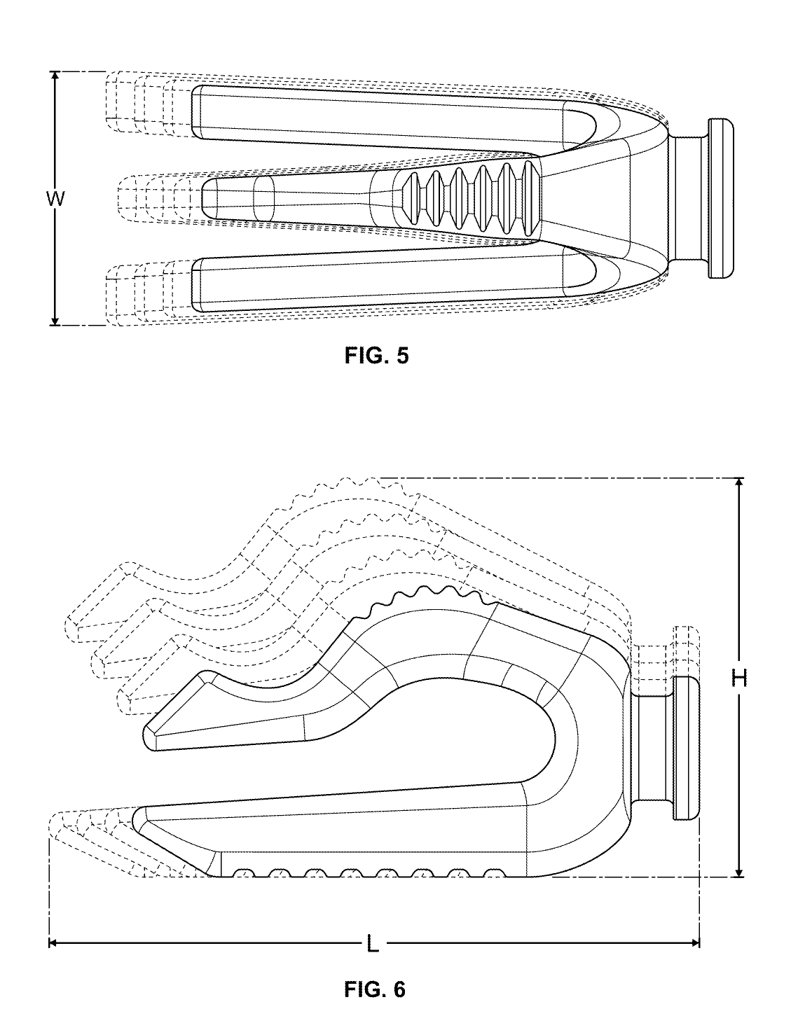

Distal surface 42 of extensions 40a, 40b may also include a plurality of ridges 44 thereon, such ridges 44 defining a sinusoidal pattern. Ridges 44 may, in one embodiment, occupy over fifty (50) percent of distal surface 42, or may occupy approximately ninety (90) percent of distal surface 42, as shown in FIGS. 2 and 4. Ridges 44 may also be formed directly into distal extensions 40a, 40b (e.g., ridges 44 may be milled into extensions 40a, 40b and/or be formed into extensions 40a, 40b via a molding or other suitable process). As with ridges 37, ridges may aid in maintaining spacer 10 in a desired position. Likewise, other structures than those shown may be employed, including knurled surfaces and surfaces including teeth.

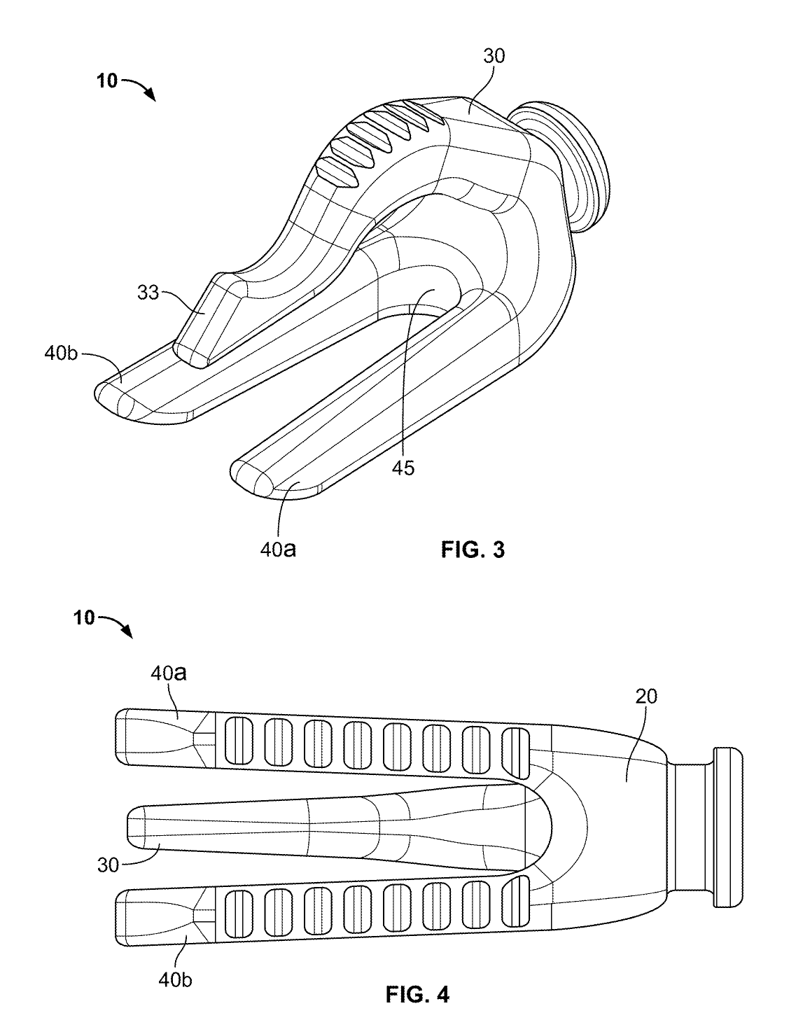

Referring now to FIG. 3, distal extensions 40a, 40b may be connected together via a curved connection 45, which traverses extensions 40a, 40b. In a particular embodiment, as shown in FIG. 4, distal extensions 40a, 40b may be connected via curved connection 45, such that extensions 40a, 40b are biased away from one another (i.e., have a non-parallel relationship). Thus, distal extensions 40a, 40b and curved connection 45 may form a “V-shape” in general. Bisecting the “V-shape” of distal extensions 40a, 40b, albeit in an elevated plane, may be proximal extension 30. Stated differently, proximal extension 30 may lie midway between distal extensions 40a, 40b, which may broadly define a “V-shape.” This is best illustrated in the view of FIG. 4.

Referring again to FIGS. 1-2, joint spacer 10 may also incorporate an anterior extension 50 comprised of a first section 51 having a first diameter 52 and a second section 53 having a second diameter 54. In a particular embodiment, second diameter 54 may be greater than first diameter 52, and first section 51 may be longer than second section 53. Sections 51, 53 may also be generally circular and may, in one embodiment, be concentric circles extending about axis 57 (e.g., circular sections 51, 53 may share the same center, designated generally as axis 57). Of course many different shapes may be employed in the design of sections 51, 53.

Anterior extension 50 may further have a substantially flat anterior surface 55 (FIG. 1) which includes an aperture 56. In a particular embodiment, aperture 56 may have a substantially square geometry and may extend a distance into extension 50; and, in a specific embodiment, this distance may extend past the combined lengths of sections 51, 53, but not completely through the body 20 of spacer 10. The substantially square geometry of aperture 56 may, in one embodiment, be designed to interact with a portion of an insertion/removal (i.e. IR) instrument, as do sections 51, 53 of extension 50. The structure and use of this IR instrument is set forth in subsequent sections. Both aperture 56 and the IR instrument 80 may be varied as far as their particular shape goes, as long as these structures are capable of cooperating with one another, such cooperation being discussed more fully below.

As shown in FIGS. 5-6, it is contemplated that joint spacer 10 may be offered in a plurality of sizes. Indeed, the range of height (“H”) for all sizes of spacer 10 may be within 0.5-1.5 inches, the range of length (“L”) for all sizes within 1.0-2.0 inches, and the range of width (“W”) for all sizes within 0.5-1.0 inches. These size offerings are based on an anthropometric study of knee joints of various patients, with the appropriate size being selected for each patient, but can be varied depending upon the particular use for spacer 10. Sizes other than those listed are also contemplated. Thus, spacer 10 is configured to accommodate differently sized knee joints, i.e., differently dimensioned intercondylar notches, different spacing dimensions between the proximal tibia and distal femur, and varying dimensions for tibial eminence.

Spacer 10, as described, may also be composed of any material suitable for temporary implantation into a patient, and may, for example, be composed of a polymeric material such as polyether ether ketone (PEEK).

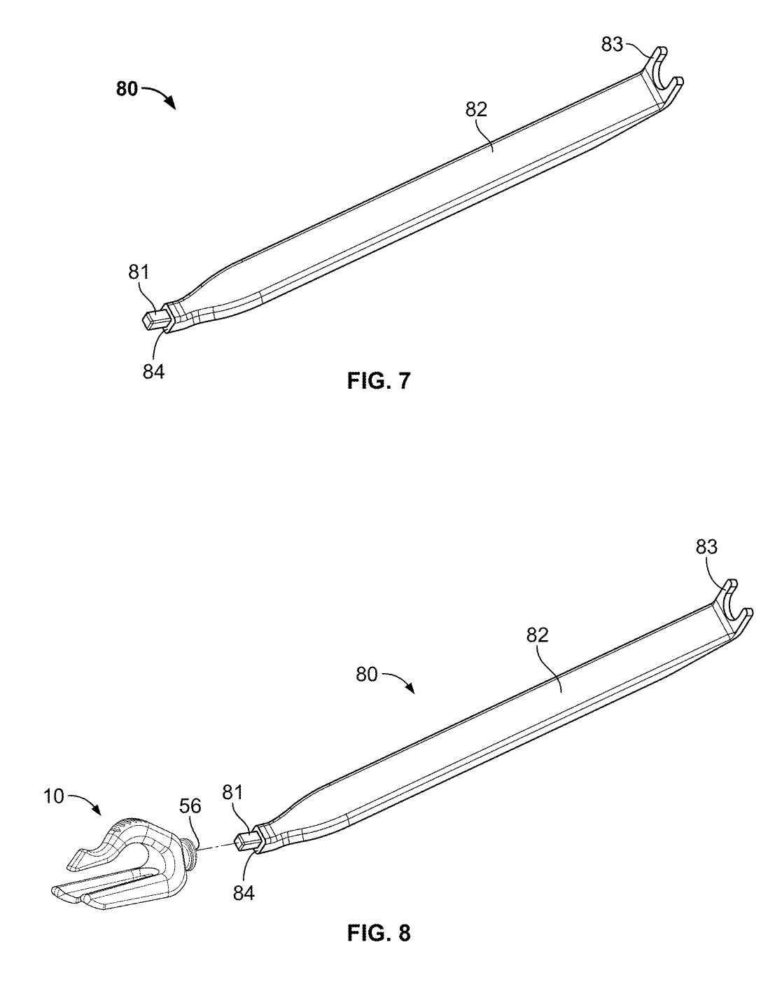

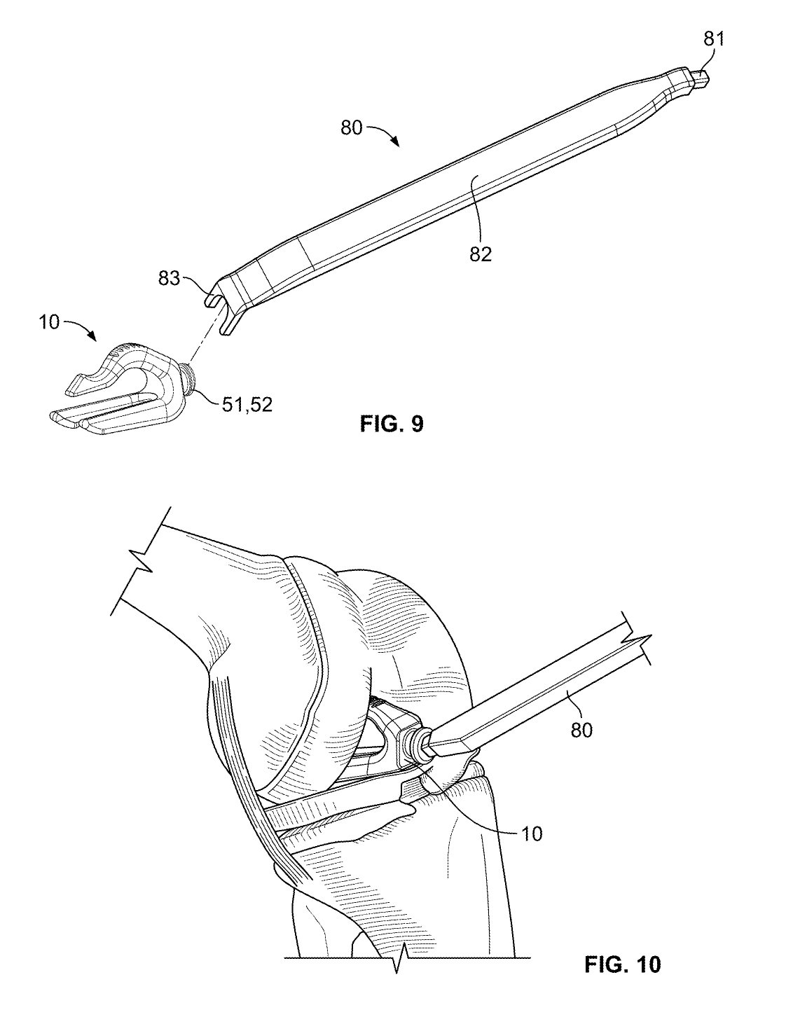

FIG. 7 illustrates an IR instrument 80 in perspective, the IR instrument 80 including an insertion geometry 81 on a first end of the instrument, a removal geometry 83 on a second end of the instrument, and an elongate connection region 82 therebetween. In a particular embodiment, insertion geometry 81 may be in the form of a post, and removal geometry 83 may be in the form of a claw (e.g., similar to that found on a claw hammer). Insertion geometry 81 may be designed to interface with aperture 56 of spacer 10 and, thus, may have a square geometry. Removal geometry 83 may be designed to interface with the first section 51 (and the first diameter 52) of anterior extension 50; and, thus, may have a semi-circular cutout for interacting with circular first section 51. In one embodiment, both insertion and removal geometries 81, 83 may be angled with respect to elongate connection region 82 to assist in engaging the same with portions of the spacer 10 (e.g., with anterior extension 50).

The interface between insertion geometry 81 and aperture 56 (e.g., a square post with a square aperture) allows for rotational control of the spacer 10 during insertion into the joint space. Insertion geometry 81 may also engage aperture 56 in anterior extension 50 at different angles, thus facilitating insertion of spacer 10 via diverse surgical approaches. Likewise, the interaction between removal geometry 83 and the first section 51 of anterior extension 50 (e.g., a semi-circular claw and a circular extension) allows for removal of spacer 10 at various angles. Stated differently, semi-circular removal geometry 81 permits three hundred and sixty (360) degree access to first section 51 of anterior extension 50, thus allowing for removal of spacer 10 via different approaches.

In a preferred embodiment, IR instrument 80 may also include ridges (not shown) on elongate connection region 82 to provided improved user grip during use. Further, it is contemplated that insertion geometry 81 may include a stop surface 84 for abutting against the flat anterior surface 55 of anterior extension 50. Thus, insertion geometry 81 may be inserted into aperture 56 in anterior extension 50 until stop surface 84 abuts flat anterior surface 55, at which point spacer 10 may be fully engaged with IR instrument 80

As alluded to above, joint spacer 10 may be inserted between the distal femur and the proximal tibia and about the ACL and PCL, potentially during a UKR or other knee arthroplasty surgery, such as bi-compartmental knee replacement or tri-compartmental knee replacement. The manner of this insertion is set forth in detail below.

First, a surgeon, nurse, or other skilled practitioner (hereinafter “the user”) may insert insertion geometry 81 of IR instrument 80 into correspondingly shaped aperture 56 in anterior extension 50 of spacer 10. The orientation of spacer 10 and IR instrument 80 during this insertion is shown in FIG. 8. Moreover, in this configuration, compression between insertion geometry 81 and aperture 56 may secure IR instrument 80 to the joint spacer 10. In particular, slight differences in dimensions between aperture 56 and insertion geometry 81 may facilitate compression therebetween (e.g., aperture 56 may be slightly smaller than insertion geometry 81, thus facilitating compression). Joint spacer 10 may therefore be manipulated via IR instrument 80 without fear of spacer 10 disconnecting from IR instrument 80.

As noted above, insertion geometry 81 may be inserted into aperture 56 in anterior extension 50 at many different positions, thus facilitating insertion of spacer 10 into a knee joint through many different surgical approaches. Once insertion geometry 81 is fully inserted within aperture 56 (e.g., upon stop surface 84 abutting against the flat anterior surface 55 of anterior extension 50), the user may manipulate spacer 10 into the knee joint of a patient.

Referring to FIG. 10, with a patient's knee joint placed in flexion (e.g., to approximately ninety (90) degrees), and with spacer 10 connected to IR instrument 80, spacer 10 may be angled such that lead-in surface 33 of proximal extension 30 is inserted between the ACL and PCL, and proximal extension 30 is disposed adjacent the distal femur. Here, the tapered shape of lead-in surface 33 may facilitate insertion of proximal extension 30 between the ACL and PCL (e.g., the tapered lead-in surface 33 may more easily slide past the distal femur). As the user advances spacer 10 in an anterior-to-posterior direction, the user may then rotate spacer 10, such that the ACL and PCL become seated between distal extensions 40a, 40b. In this configuration, proximal extension 30 may be disposed between the ACL and PCL, while distal extensions 40a, 40b may surround, or be positioned adjacent, the ACL and PCL. Further, distal extensions 40a, 40b may be situated adjacent the proximal tibia, with ridges 44 contacting tibial bone.

Alternately described, a user may orient the axis 57 of spacer 10 generally vertical as spacer 10 approaches the knee joint of a patient. Upon advancing spacer 10 in an anterior-to-posterior direction, the user may insert proximal extension 30 between the ACL and PCL. Then, the user may orient axis 57 substantially horizontal while rotating spacer 10 about axis 57 to capture the ACL and PCL between distal extensions 40a, 40b. Upon further advancement of spacer 10 in the anterior-to-posterior direction, cradle region 34 of proximal extension 30 may receive the posterior region of the intercondylar notch of the femoral bone therein. Likewise, as spacer 10 is advanced in the anterior-to-posterior direction, distal extensions 40a, 40b may be positioned on the proximal tibia, with ridges 44 engaging tibial bone. Thus, the distal femur and the proximal tibia may be stabilized and separated via spacer 10 by a distance between cradle region 34 and distal extensions 40a, 40b. Again, in this configuration, proximal extension 30 may be disposed between the ACL and PCL, while distal extensions 40a, 40b may surround, or be positioned adjacent, the ACL and PCL.

During insertion of spacer 10, as described, proximal extension 30 may bend under insertion loads and then “spring back” into position when the posterior region of the intercondylar notch rests within cradle region 34 of proximal extension 30. Thus, due to the flexible characteristics of proximal extension 30, spacer 10 may be more easily inserted between the proximal tibia and the distal femur during a UKR (or other knee replacement surgery). Further, upon insertion of spacer 10 between the proximal tibia and the distal femur, a tactile sensation will alert the user when spacer 10 is advanced by the proper amount. Specifically, the user may distinctly feel when the posterior region of the intercondylar notch engages flat region 36 of proximal extension 30, thus signaling proper and full insertion of spacer 10. Further, the curvature of cradle region 34 may prevent over and/or under insertion of spacer 10 within the knee joint. As an example, were spacer 10 to move posteriorly into the knee joint, the curvature of cradle region 34, and specifically flat region 36 adjacent such region 34, may prevent movement of spacer 10 beyond a desired point (e.g., past the intercondylar notch of the femur).

FIG. 10 illustrates spacer 10 being fully inserted between the proximal tibia and distal femur of a knee joint, as described above. Once inserted in this manner, the user may move the knee joint in flexion and extension, as needed. Further, the proximal tibia and the distal femur may be prepared through the use of various cutting instruments, as in traditional knee replacement surgeries, with spacer 10 inserted in the joint. During such preparation of bone, spacer 10 may provide stability to the knee joint, e.g., maintain the spacing between the proximal tibia and the distal femur. In this way, spacer 10 may prevent the femoral and tibial bones from collapsing onto one another as bone and/or tissue is removed from the surgical site, i.e., the knee joint.

Moreover, since IR instrument 80 is removable from spacer 10, the construct does not interfere with the preparation of the tibia and femur during surgery (e.g., the tools used for preparation of the knee joint do not bump into a portion of spacer 10). Stated differently, as nothing is projecting from spacer 10 once the same is inserted into the knee joint, the working area of the cutting tools used is not interfered with. Accordingly, spacing and stabilization between the femur and tibia is maintained, with no appreciable interference in the preparation of such bones during surgery.

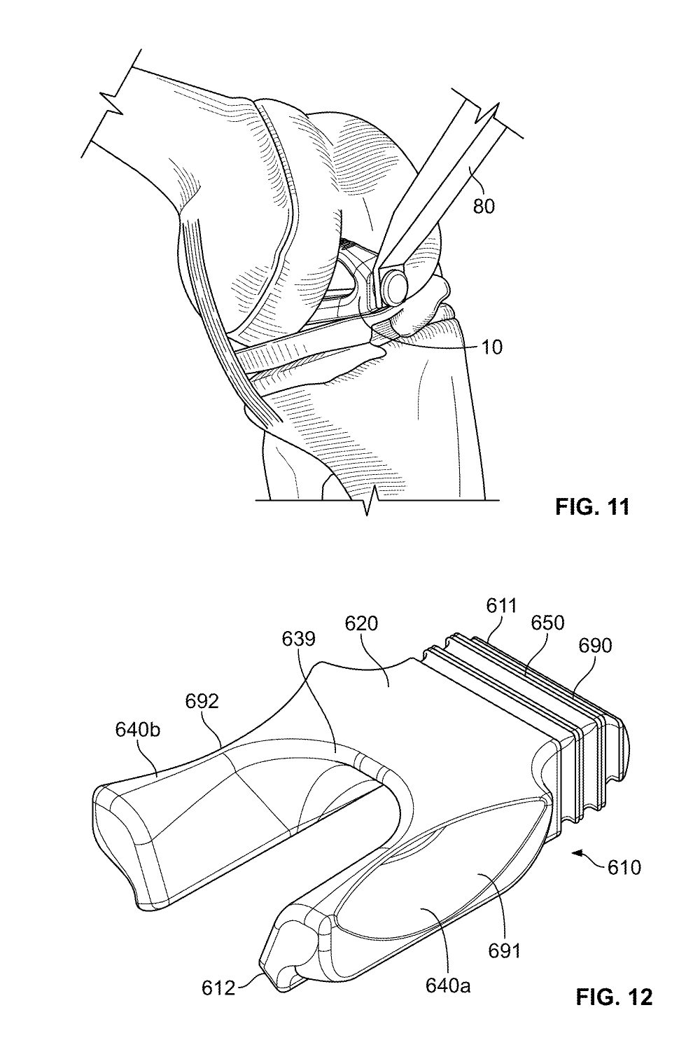

Following bone and/or tissue preparation, and potentially after fixation of a prosthesis to the distal femur and/or the proximal tibia (or alternatively, before), removal of spacer 10 may be consistent with that previously described. Specifically, as shown in FIG. 11, removal geometry 83 of IR instrument 80 may engage the first section 51 of anterior extension 50 (e.g., the open semi-circular portion of removal geometry 83 may partially surround the first diameter 52 of first section 51). In this configuration, the user may grasp connection region 82, which, in several embodiments may be an elongate handle, and pull longitudinally. This may cause removal geometry 83 to abut against second section 53 of anterior extension 50. Due to the increased diameter 54 of second section 53 relative to first section 51, pulling longitudinally in the manner described above may facilitate removal of spacer 10 from the patient's knee joint. Again, as with the connection between insertion geometry 81 and aperture 56, here, removal geometry 83 may engage first section 51 of anterior extension 50 at many different positions; thus, IR instrument 80 may be configured to remove spacer 10 via several different surgical approaches.

Upon engaging removal geometry 83 with anterior extension 50 and pulling longitudinally, cradle region 34 of proximal extension 30 may slide past the intercondylar notch of the patient's femur, and distal extensions 40a, 40b may slide in a posterior-to-anterior direction along the tibia. Subsequently, once cradle region 34 and distal extensions 40a, 40b are fully disengaged with the distal femur and proximal tibia, respectively, spacer 10 may be removed from contact with bone altogether and discarded or reused, as appropriate. At this point, the knee surgery may be complete, that is, provided the prosthetic implant has already been implanted.

Consistent with that described in relation to FIGS. 5-6, it is contemplated that spacer 10 may be offered in various sizes to accommodate a variety of joint geometries; and that, for example, a kit may be offered which includes multiple spacers 10 of different sizes and an IR instrument 80. In one embodiment, three (or any number) different sized spacers 10 may be packaged with an IR instrument 80. In this embodiment, all of the components within the kit may be designed for a single-use surgery, and may be made of any of the materials previously described. Thus, the components of the kit may be disposed of following surgery. The variety of spacers and IR instruments offered with this kit may also have any of the features of spacer 10 and IR instrument 80, as hereinbefore disclosed. Thus, a kit may be provided, which allows a surgeon to select an appropriately sized spacer 10 and IR instrument 80 for the UKR (or other surgery) to be performed. The surgeon may select one of the differently sized spacers 10 according to the spacing required between the tibia and femur of a particular patient. Thus, the natural spacing for knee joints of varying sizes and shapes may be accommodated.

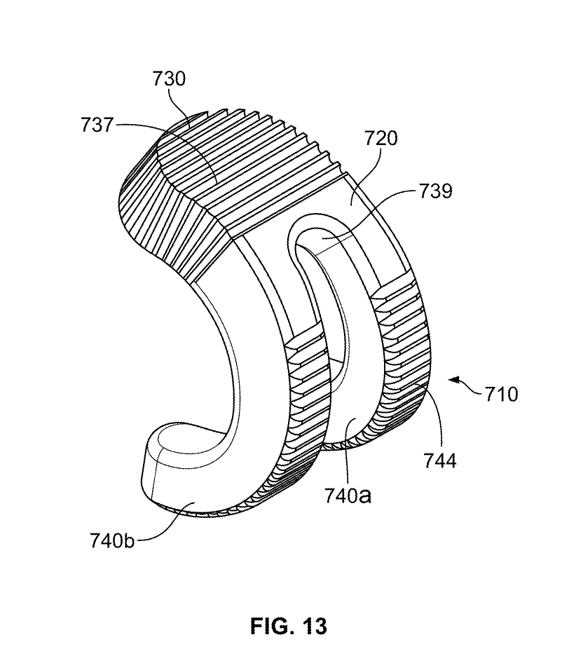

Alternate embodiments of spacer 10, as shown in FIGS. 12-13, are also contemplated. The first of these embodiments, as shown in FIG. 12, includes a spacer 610 having a body 620 with an anterior end 611 and a posterior end 612. Extending from the body 620 in an anterior-to-posterior direction may be a pair of extensions 640a, 640b. Extending in a direction opposite extensions 640a, 640b may be an anterior extension 650, which may have a series of serrations 690 formed thereon. Extensions 640a, 640b may be spaced apart from one another and may be connected via a curved region 639. Like spacer 10, spacer 610, in this embodiment, may be inserted within the knee of a patient, such that extensions 640a, 640b surround the ACL and PCL to protect such ligaments during bone preparation and/or resection during a knee replacement surgery. Specifically, spacer 610 may be engaged with an insertion instrument, or may be manually grasped via serrations 690, and subsequently inserted into the knee joint of a patient, such that extensions 640a, 640b are positioned on adjacent sides of the ACL and PCL. Cup-shaped features 691, 692 may also be provided on extensions 640a, 640b of spacer 610 for cradling the condyles of the femur near the intercondylar notch. These cup-shaped features 691, 692 may also allow flexion and extension of a knee while spacer 610 is being inserted. Accordingly, spacer 610 may provide many of the benefits afforded by spacer 10, although utilizing different structure.

The second of these alternate embodiments, as shown in FIG. 13, includes a spacer 710 having a body 720 with a proximal extension 730 and a pair of distal extensions 740a, 740b. Distal extensions 740a, 740b may be spaced apart from one another and may be connected together by a curved region 739 at one end. Ridges 737, 744 may be formed, respectively, on proximal extension 730 and distal extensions 740a, 740b. In use, much like spacer 10, spacer 710 may be inserted between the distal femur and proximal tibia of a patient, such that proximal extension 730 abuts the distal femur, and distal extensions 740a, 740b abut the proximal tibia. Moreover, once inserted, distal extensions 740a, 740b may surround the ACL and PCL so as to protect such ligaments during bone preparation in a UKR (or other) knee arthroplasty surgery.

In each of the alternate embodiments (FIGS. 12-13) noted above, spacers 610, 710 may maintain adequate spacing and/or joint stabilization between the femur and the tibia during a UKR, knee arthroplasty, or other surgery. Thus, spacers 610, 710 may be used for substantially the same purposes as spacer 10, although configured differently.

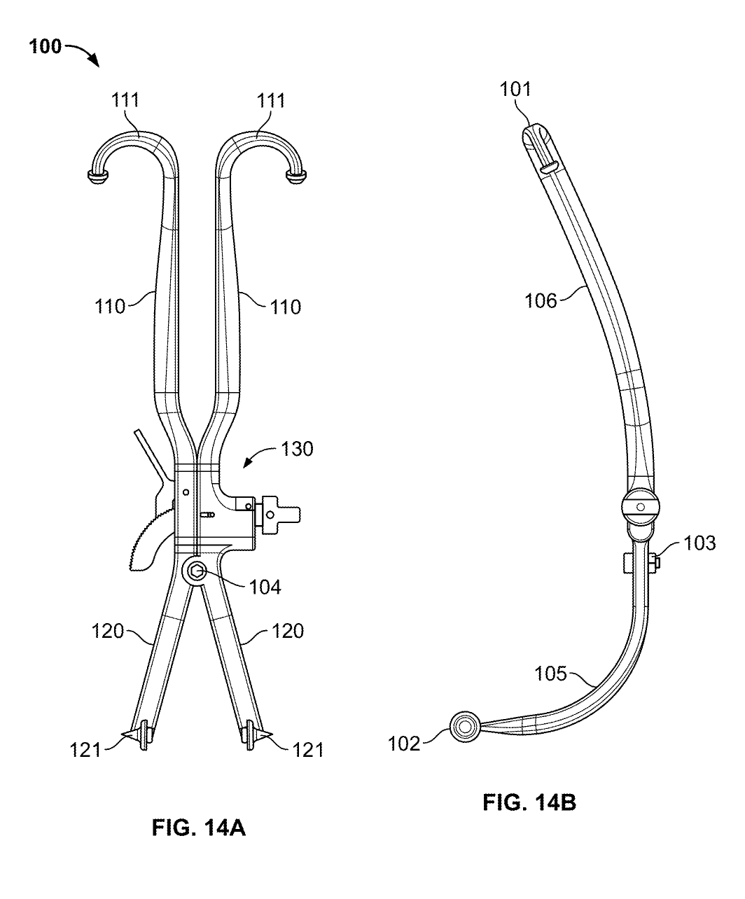

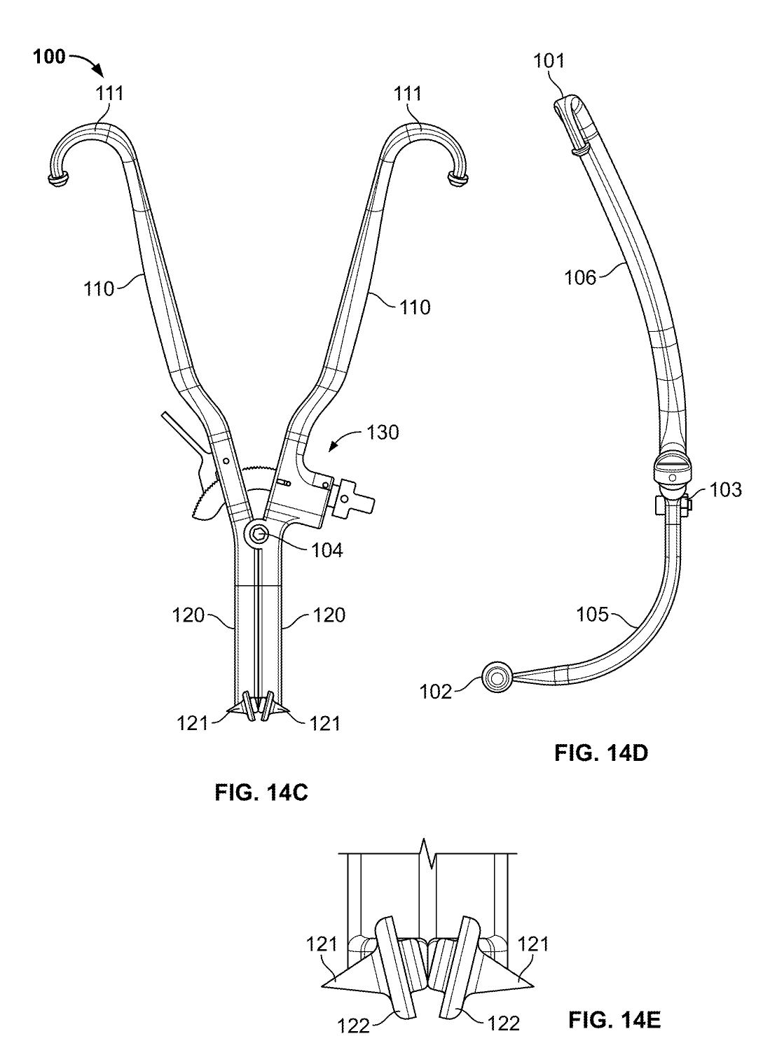

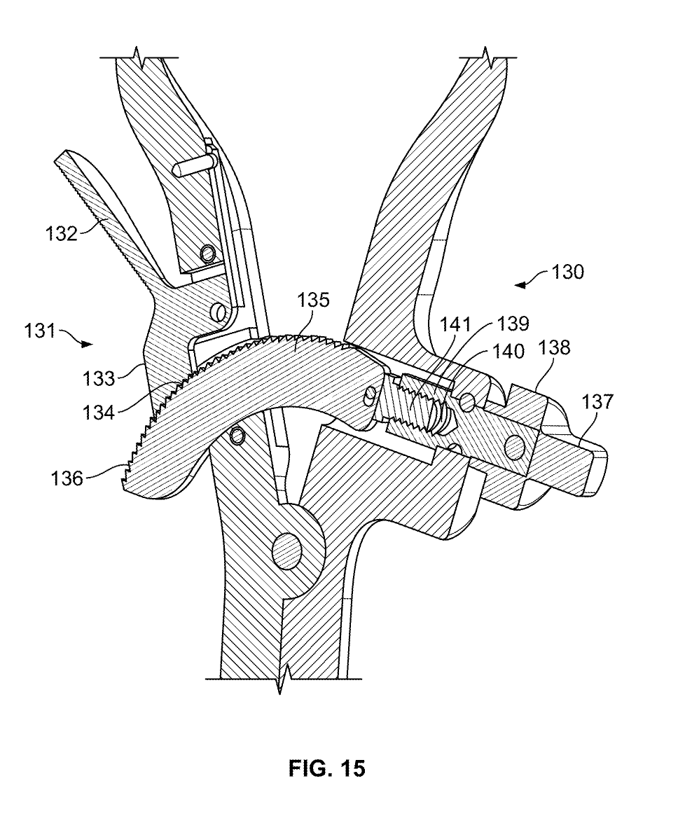

Further mechanisms for distracting and/or maintaining the space between the proximal tibia and distal femur are disclosed in FIGS. 14A-E. In particular, in one embodiment of the present invention, a distractor 100 is provided for distracting and/or maintaining the space between the proximal tibia and distal femur, for example, during a knee arthroplasty procedure. Distractor 100, as shown in FIGS. 14A-E, may include a first end 101 having a set of handles 110 and a second end 102 having a set of arms 120 with projections 121 thereon. Handles 110 may be connected together at a screw-and-nut configuration 103, thus allowing handles 110 to rotate or pivot about a point 104.

Handles 110 and arms 120 may also be angled with respect to one another, such that, in one configuration (FIG. 14A), arms 120 are spaced apart from one another and, in another configuration (FIG. 14C), arms 120 are disposed adjacent one another. The former of these orientations (FIG. 14A) may be achieved via a user squeezing handles 110 towards one another. Thus, distractor 100 is configured so that, when handles 110 are situated apart from one another (FIG. 14C), arms 120 may be disposed adjacent one another, and vice versa (FIG. 14A).

As also shown in FIGS. 14B and 14D, the transition between handles 110 and arms 120 may form a curved region 105, such that the second end 102 of distractor 100 defines a hook when viewed from the side. Moreover, first end 101 of distractor may likewise include a curved region 106; although, in one embodiment, such region 106 may have a radius of curvature, which is slightly less than that of curved region 105. First end 101 of distractor 100, and in particular each of handles 110 at first end 101, may also include a hooked portion 111, as shown best in FIGS. 14A and 14C, for connecting to a separate component of a bone preparation system (not shown), such as a leg positioner or strap (also not shown).

Referring now to FIG. 14E, projections 121, as noted above, may be formed on a portion of each arm 120 of distractor 100. Projections 121 may also include a flange 122 extending about an end thereof for protecting projections 122 from sensitive tissue upon insertion (e.g., from the PCL and ACL once distractor 100 is inserted into the knee, as detailed below). Further, when arms 120 are disposed adjacent one another (FIG. 14C), each projection 121 may be angled with respect to the other. In some embodiments, this angle may be about thirteen and one-half (13.5) degrees. Yet, when arms 120 are biased away from one another (FIG. 14A), projections 121 may not be angled with respect to one another. In other words, upon full distraction of distractor 100 (FIG. 14A), a straight axis may run directly through the tip of both projections 121.

A distraction mechanism 130 may also be provided with distractor 100. As shown in FIG. 15, in one embodiment, this mechanism 130 may include a ratchet configuration 131. In particular, ratchet configuration 131 may contain a lever 132 having, at one end 133, a saw-toothed surface 134. Further, extending from a portion of distraction mechanism 130 may be a post 135 having a correspondingly saw-toothed surface 136 that interacts with saw-toothed surface 134. Lever 132 may also be biased towards post 135, such that saw-toothed surface 134 may be constantly in contact with saw-toothed surface 136, that is, unless lever 132 is actuated. Individual teeth on saw-toothed surface 134 of lever 132 may be angled in one direction, while individual teeth on saw-toothed surface 136 of post 135 may be angled in an opposing direction, such that movement of surface 134 with respect to surface 136 may proceed in only one direction. To release ratchet configuration 131, after distracting distractor 100 through a squeezing action, one may simply actuate or depress lever 132 causing saw-toothed surfaces 134, 136 to disengage.

If, upon distracting distractor 100 a particular amount, a user wishes to fine tune the amount of distraction or separation between projections 121, the user may utilize a fine adjustment mechanism 137 provided with distractor 100. In one embodiment, fine adjustment mechanism 137 may comprise a knob 138 having a post 139 with an internally threaded bore 140 for engaging an externally threaded portion 141 of post 135. In use, rotation of knob 138 may cause a corresponding rotation of post 139 and internally threaded bore 140. This rotation, due to the interaction of internally threaded bore 140 and externally threaded portion 141, may cause post 135 of distraction mechanism 130 to be drawn within bore 140 in a direction towards knob 138. Likewise, rotating knob 138 in an opposing direction may cause post 135 of distraction mechanism 130 to move outward of bore 140 and in a direction away from knob 138, that is, if lever 132 is in the actuated position. In this manner, a user may use fine adjustment mechanism 137 to more precisely dial-in the amount of distraction desired.

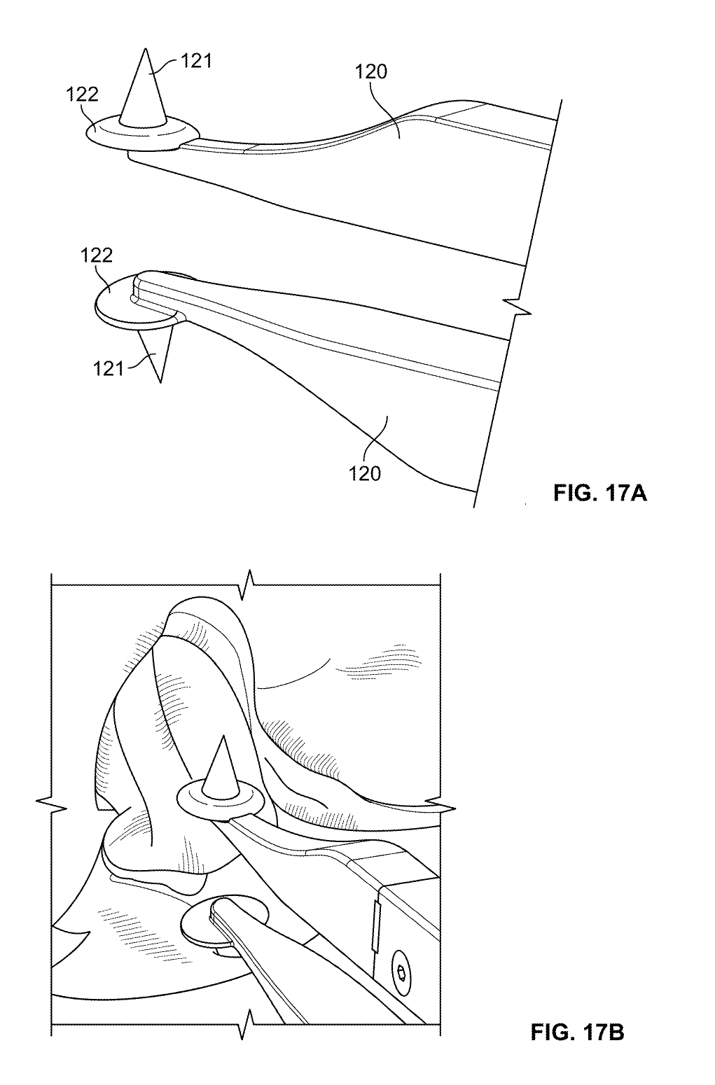

In use, distractor 100 may be inserted between the proximal tibia and the distal femur of a patient to separate and/or maintain the spacing between the bones. In particular, as shown in FIGS. 17A-B, one of projections 121 may be inserted within the knee cavity of a patient; and, specifically, such projection 121 may be disposed in the intercondylar notch region of the femur. Moreover, an opposing projection 121 may be inserted into a portion of the tibia. Each of projections 121 may then be embedded into bone, via distraction of distractor 100. In other words, a user may squeeze handles 110 of distractor 100, thus causing each of projections 121 to contact bone and become embedded therein after further actuation of handles 110. As discussed above, upon separation of arms 120 of distractor 100, via squeezing of handles 110, ratchet configuration 131 may maintain arms 120 in their separated condition so as to maintain the spacing therebetween. In particular, saw-toothed surface 136 on post 135 may engage with saw-toothed surface 134 on lever 132, thus precluding inadvertent loss of separation between arms 120.

With distractor 100 inserted in the knee of a patient, as described, preparation of the proximal tibia and distal femur may take place (e.g., through preparation of bone carried out via one or more cutting tools). During such preparation, distractor 100 may maintain the necessary space between the proximal tibia and distal femur, and ensure that the joint space does not collapse. Then, a prosthetic implant (not shown) may be attached to the prepared bone surface(s) to repair the diseased and/or damaged surface(s), as is known in traditional knee arthroplasty procedures. Subsequently (or alternatively before, if desired), the user may actuate lever 132 allowing for the release of ratchet configuration 131 and for removal of distractor 100 from the patient, as needed.

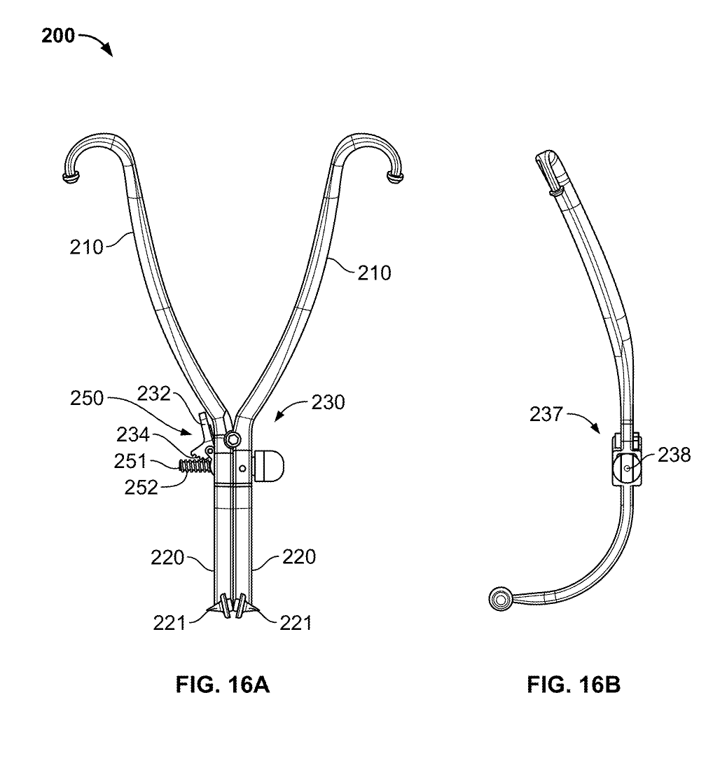

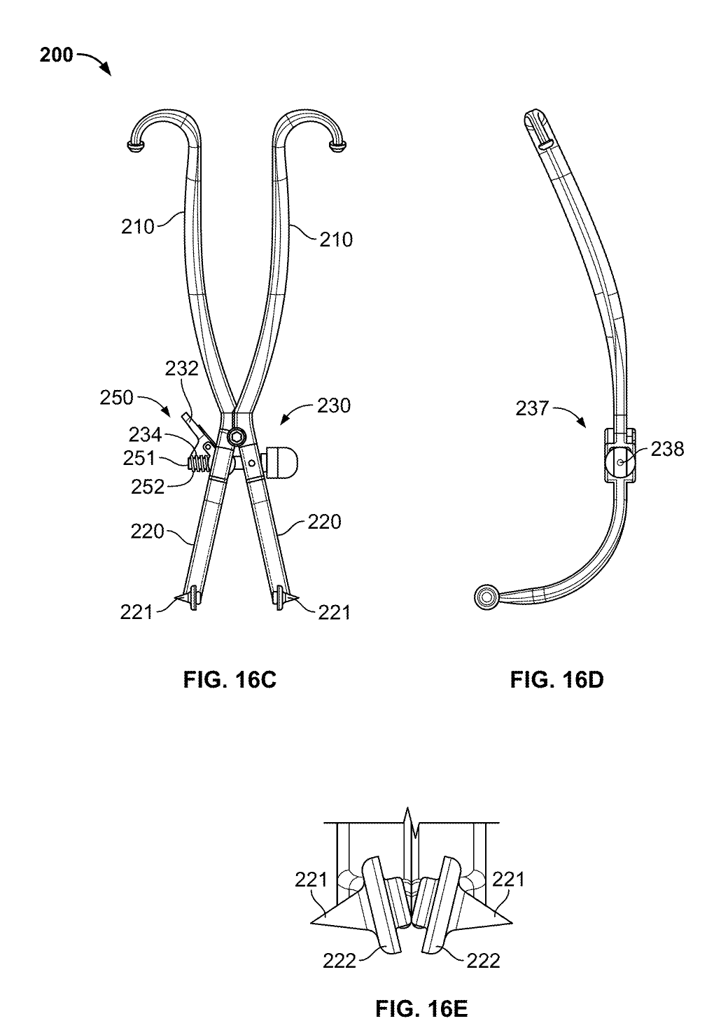

An alternate version of distractor 100 is shown in FIGS. 16A-16E. Here, a distractor 200 is provided with many of the same features as that found with distractor 100 (e.g., handles 210, arms 220, projections 221, etc.) Accordingly, except where provided in this embodiment, like reference numerals refer to like elements.

One difference between distractor 100 and distractor 200 is the inclusion of a distraction mechanism 230 in the form of a buttress release mechanism 250. In particular, buttress release mechanism 250 may include a post 251 having a series of external threads 252 at one end. Further, the post 252 may extend through respective apertures (not shown) in a portion of each arm 220; and the external threading 252 on post 251 may cooperate with a saw-toothed surface 234 formed on a lever 232 of the buttress release mechanism 250. In one embodiment, lever 232 may be out of engagement with the external threading 252 on post 251 when arms 220 are disposed adjacent one another (FIG. 16A), and lever 232 may be engaged with external threading 252 when arms 220 are separated from one another (FIG. 16C). Thus, upon distracting distractor 220, buttress release mechanism 250, and in particular saw-toothed surface 234 on lever 232 thereof, may be actuated to engage with threading 252 on post 251 to maintain distractor 200 in its distracted orientation (FIG. 16C).

Distractor 200 may further include a fine adjustment mechanism 237 having a knob 238. Knob 238 may be connected to post 251 of buttress release mechanism 250, such that rotation of knob 238 may cause rotation of post 251. As such, with distractor 200 in a partially distracted orientation, and with lever 232 engaged with post 251, knob 238 may be rotated so as to cause corresponding movement of arms 220. In particular, as knob 238 is rotated in one direction (e.g., clockwise), arms 220 may move away from one another, and as knob 238 is rotated in an opposite direction (e.g., counterclockwise), arms 220 may move closer to one another. The interaction between saw-toothed surface 234 and threading 252 on post 251 facilitates this motion. Thus, once inserted within the knee cavity (or other surgical site) of a patient, fine adjustment mechanism 237 of distractor 200 may be used to precisely position arms 220 of distractor 200.

To release distractor 200 from its distracted condition, one may simply actuate lever 232 (e.g., pull on such lever 232) and cause saw-toothed surface 234 to disengage external threading 252 on post 251.

Distractor 200 may be used in the same manner as distractor 100, as discussed with reference to FIGS. 17A-B; and thus, such use is not detailed here.

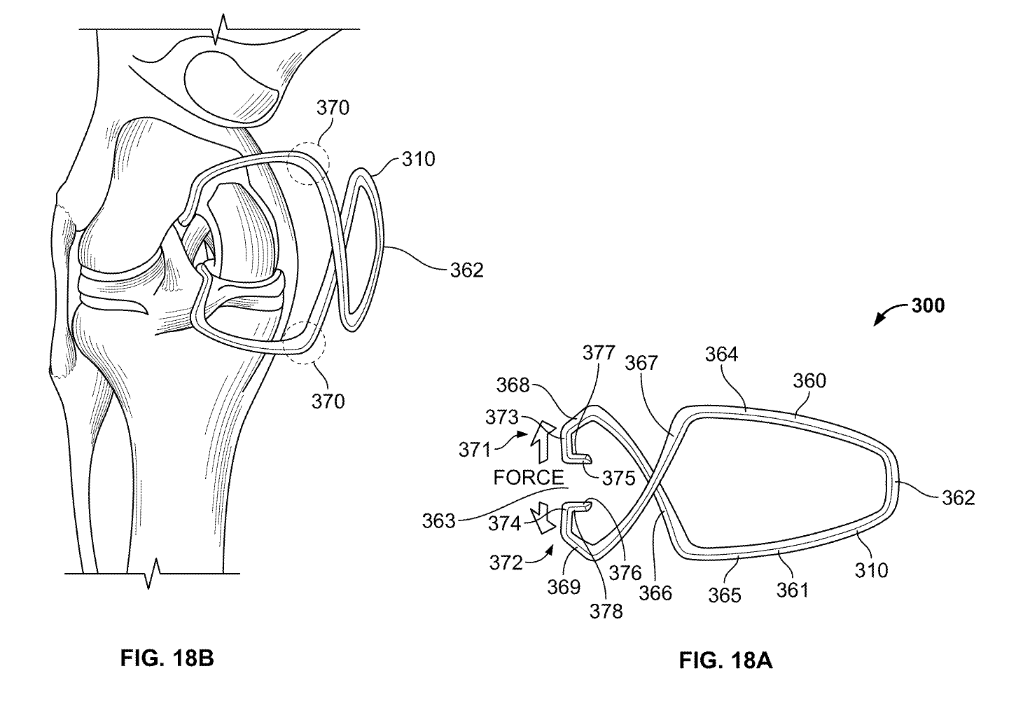

Referring now to FIGS. 18A-B, another embodiment distractor is shown. Here, a distractor 300 is provided and comprises a handle portion 310 in the form of a set of spaced apart rods 360, 361. Such rods 360, 361 may be connected at one end 362 of the distractor 300 and may be separated at an opposing end 363. Moving in a direction from end 362 to opposing end 363, rods 360, 361 may cross or overlap one another. Stated differently, each rod 360, 361 may include a first section 364, 365, respectively, and a second section 366, 367, such that the first section 364, 365 is angled with respect to the second section 366, 367. Moreover, in one embodiment, this angle may be such that the second section 366, 367 of rods 360, 361 intersect one another.

Rods 360, 361 may also include a third section 368, 369, which may have a curvature 370 with respect to the first 364, 365 and second 366, 367 sections. In other words, in a particular embodiment, first 364, 365 and second 366, 367 sections may lie in relatively the same first plane, and third section 368, 369 of rods 360, 361 may lie in a different second plane, such planes being angled or having a curvature 370 with respect to one another. In some embodiments of distractor 300, this angle or curvature 370 may be roughly ninety (90) degrees. Thus, when viewed from the side, the angle or curvature 370 between, collectively, first 364, 365 and second 366, 367 sections, and third section 368, 369 may be “L-shaped.” This allows a user to position distractor 300 out of the space required for the surgical procedure (e.g., knee arthroplasty), as distractor 300 may conform to the anatomy of the patient via curvature 370 of rods 360, 361 (FIG. 18B).

At end 363 of rods 360, 361 there may also be formed respective hook portions 371, 372 for contacting a portion of the proximal tibia and distal femur, as shown in FIG. 18B. To accommodate the particular curvature 370 of rods 360, 361 such hook portions 371, 372 may be configured as follows—each hook portion 371, 372 may comprise a first segment 373, 374 extending at an angle with respect to third section 368, 369 of rods 360, 361 (e.g., in FIG. 18A, generally towards end 362); a second segment 375, 376 may terminate each hook 371, 372 and may be angled with respect to the first segment 373, 374 (e.g., in FIG. 18A, generally away from handle portion 310); and bone-contacting surfaces 377, 378 may be situated on second segments 375, 376 for contacting bone (e.g., a portion of a proximal tibia or a portion of a distal femur, such as, for example, an intercondylar notch thereof).

In use, after a user sufficiently compresses or squeezes handle portion 310 of distractor 300, thus causing movement of hook portions 371, 372 toward one another, distractor 300, and in particular bone-contacting surfaces 377, 378, may be positioned against a surface of bone (e.g., a portion of a proximal tibia and distal femur, respectively). Due to the connection between rods 360, 361 at end 362, bone-contacting surfaces 377, 378 may then be biased or moved away from one another, thus causing distraction of the space between the proximal tibia and distal femur. In this manner, the user may maintain the spacing between the proximal tibia and distal femur (or other joint in which distractor 300 is placed) during bone preparation. Further, due to curvature 370, handle portion 310 of distractor 300 may be situated away from the surgical space, and consequently, may decrease and/or eliminate interference with instruments used in the preparation of bone. For example, in the preparation of a medial condyle of a femur, distractor 300 may be positioned within the joint space, such that handle portion 310 resides on a lateral portion of the knee. Accordingly, the joint may be distracted, yet distractor 300 may not interfere with preparation of the medial condyle through the use of the various cutting instruments previously described. Likewise, during preparation of a lateral condyle, distractor 300 may be positioned within the joint space, such that handle portion 310 resides on a medial portion of the knee. Thus, distractor 300 may be situated in a variety of positions, so as to minimize or eliminate interference with bone preparation during surgery.

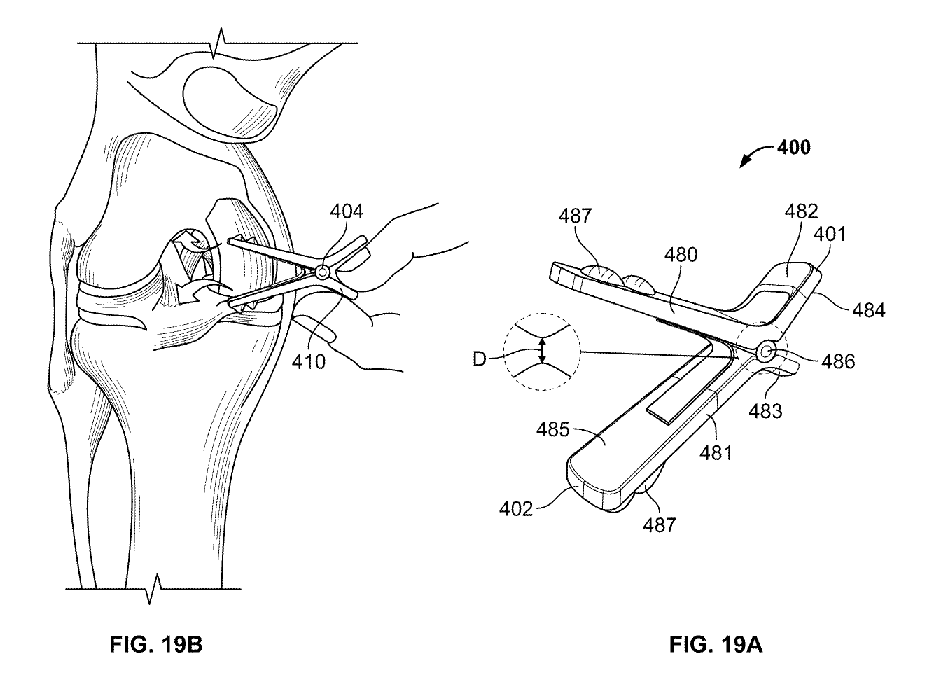

Referring now to FIGS. 19A-B, a variant of the afore-described distractors, distractor 400, is shown. Distractor 400 may include a first end 401 and a second opposing end 402. Further, situated adjacent first end 401 may be a handle or finger-gripping portion 410, as shown in FIG. 19B, for manipulating distractor 400. Distractor 400 may also include opposing proximal 480 and distal 481 portions for engaging, respectively, different surfaces of bone (e.g., for proximal portion 480—the intercondylar notch of a femur; for distal portion 481—a portion of the proximal tibia). Proximal 480 and distal 481 portions of distractor 400 may also, in one embodiment, be curved or angled, such that the respective portion 480, 481 is contoured to the intended bone-contact surface. In a particular embodiment, outside surfaces 482, 483 of proximal 480 and distal 481 portions, respectively, may be concave, while inside surfaces 484, 485 of proximal 480 and distal 481 portions may be convex.

As shown in detail in FIG. 19A, proximal 480 and distal 481 portions of distractor 400 may also be connected together via a spring 486. In particular, the apex of inside surfaces 484, 485 of proximal 480 and distal 481 portions may be joined together via spring 486, thusly allowing proximal 480 and distal 481 portions of distractor 400 to pivot about a point 404. In one embodiment, spring 486 may be configured such that proximal 480 and distal 481 portions are biased to remain in one position, and not flex out of such position. Stated differently, spring 486 may inhibit flexion of proximal portion 480 towards distal portion 481, and vice versa. In this way, once distractor 400 is inserted into a joint of a patient, distractor 400 may stabilize the joint and limit unwanted movement thereof.

In some embodiments of distractor 400, various features may be included therewith, such as ridges and/or bumps 487 on outside surfaces 482, 483 for creating a better contact surface with bone. Additionally, outside surfaces 482, 483 may be covered with a material (e.g., rubber) for providing traction with bone.

Distractor 400 may also, in a particular embodiment, include proximal 480 and/or distal 481 portions, which are composed of a plastic material(s) that is injection molded to the specific shape of such portions 480, 481.

In use, distractor 400, much like the previously described distractors 100, 200, 300, may be inserted within the knee joint of a patient and left therein during bone preparation. In particular, handle or finger-gripping portion 410 may be grasped by a user and distractor 400 may be manipulated between the proximal tibia and the intercondylar notch of the femur. Once so inserted, the spacing between such bones may be maintained by distractor 400 according to the distance D between the apexes of curvature of each of outside surfaces 482, 483. Additionally, distractor 400 may be resistant to over and/or under insertion, since the curvature of outside surfaces 482, 483 of proximal 480 and distal 481 portions will facilitate insertion and resist over insertion. As an example, due to the curvature of outside surface 482, the trailing end 401 of distractor 400 may abut a portion of the intercondylar notch of the femur prior to over-insertion of the distractor 400. Likewise, the curvature of outside surface 482 creates a tendency for distractor 400 to seat within the joint space at a particular location (e.g., with the intercondylar notch of the femur resting along such curvature).

Once bone preparation is complete, distractor 400 may be removed from the joint space by simply grasping handle/finger-gripping portion 410 and manipulating distractor 400 out of the joint space.

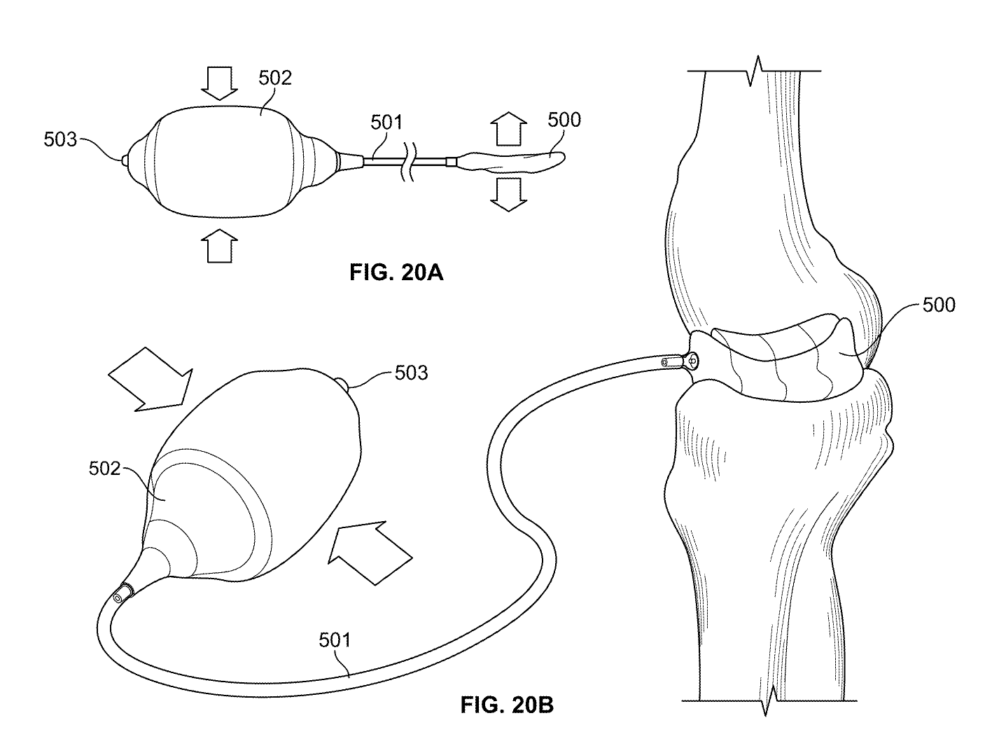

Several alternate methods for maintaining the spacing between a joint in a patient, and the associated devices used therewith, are also contemplated by the present invention, as shown in FIGS. 20A-B and 21.

Referring to FIGS. 20A-B, there is shown the use of inflatable technology for maintaining the spacing between the joint of a patient (e.g., the spacing between the tibia and femur during knee arthroplasty). Here, an inflatable member 500 is shown having, connected thereto, an air and/or fluid line 501. Line 501 may be connected to a pump 502 having a valve member 503. Pump 502 may be manually or electronically operated.

In operation, inflatable member 500 may be inserted into the joint space (e.g., between the proximal tibia and distal femur), and may be thereafter expanded using pump 502. Once expanded to a desired level, inflatable member 500 may serve to maintain adequate spacing between the joint bones, thus allowing preparation of the same without the fear of collapse of the joint space. After preparation of the bone, valve member 503 of pump 502 may be actuated to deflate inflatable member 500. Inflatable member 500 may then be removed from the joint space, whereupon further surgical procedures may take place (e.g., insertion of a prosthetic implant within or on the joint).

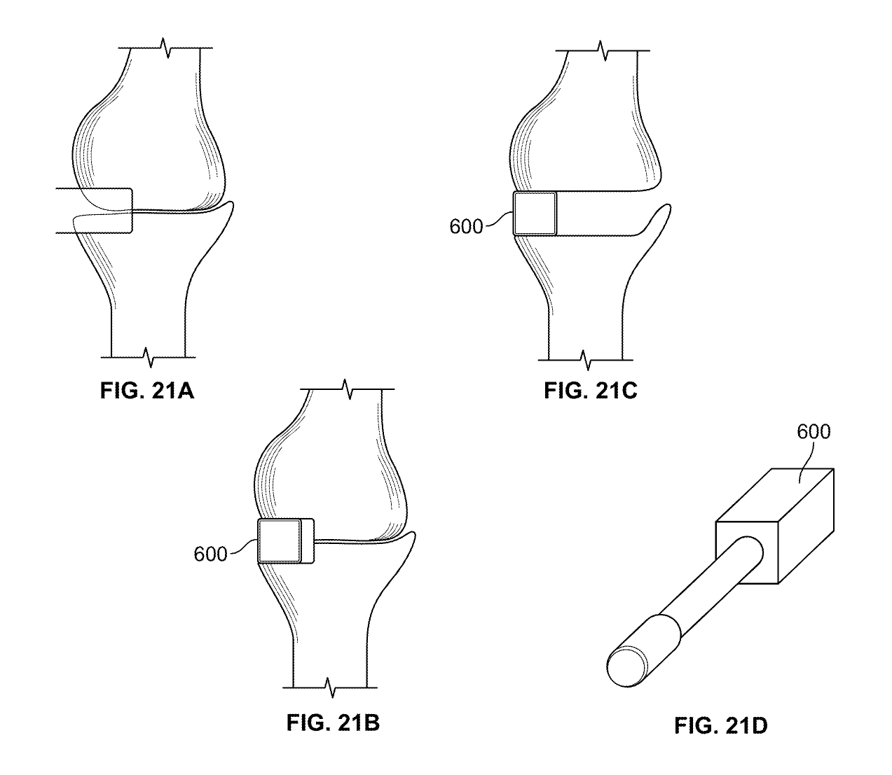

Referring now to FIGS. 21A-D, a method of preparing a joint space, and in particular, a compartment of a knee joint (e.g., either a medial and/or lateral unicondylar compartment) is disclosed. As a first step, a surgeon may prepare or resect, at least partially, a surface of one condyle of a knee joint (shown schematically in FIG. 21A). This may involve, for example, resecting a surface of the proximal tibia and a corresponding surface of one condyle of the femur. Subsequently, the surgeon may occupy the resected space with a shim or spacer 600, as shown in FIG. 21B. The shim or spacer 600 may serve to maintain the natural anatomical spacing between the joint (e.g., between the tibia and femur) during resection of the remaining surface(s) of bone. In a particular embodiment, with shim or spacer 600 inserted into the initially resected surface(s) of bone, the surgeon may engage in resection of the remaining surface(s), such as, for example, the remaining portion(s) of the condyle of the femur and the corresponding surface(s) of the tibia, through use of the cutting instruments described. This is shown schematically in FIG. 21C. Shim or spacer 600 may therefore allow the surgeon to prepare the remaining surface(s) of bone without interference, and without the joint collapsing onto itself. In specific embodiments, the shim or spacer 600 may provide a spacing of anywhere between ten (10) and thirteen (13) or more millimeters.

In the devices shown in the figures, particular structures are shown as being adapted for use in a knee arthroplasty, or other similar procedure. The invention(s) also contemplates the use of any alternative structures for such purposes, including structures having different lengths, shapes, and/or configurations. For example, while proximal extension 30 of spacer 10 has been described as being “S-shaped”, the shape and geometry of extension 30 may be varied, provided that extension 30 is configured to seat a portion of the intercondylar notch of a femur therein. In other words, it is contemplated that proximal extension 30 may be of any shape, such as, for example, an open rectangular shape, a “V-shape”, or simply straight, provided that extension 30 is configured to abut the intercondylar notch of a knee.

Likewise, although the position of proximal extension has been described as bisecting the space between distal extensions 40a, 40b, proximal extension 30 may lie at any point between distal extensions 40a, 40b. Thus, it is not essential for proximal extension 30 to lie midway between distal extensions 40a, 40b, and extension 30 may lie at any point therebetween.

As another example, in alternate embodiments of joint spacer 10, it is contemplated that ridges 37, 44 may be covered with a material providing for increased protection between the spacer 10 and bone, such as rubber or other biocompatible materials. This coating may be applied to ridges 37, 44 on proximal 30 and distal 40a, 40b extensions during or post manufacture of spacer 10. Alternatively, ridges 37, 44 may be omitted altogether (e.g., proximal 30 and distal 40a, 40b extensions may not have ridges at all).

Even further, while lead-in surfaces 33, 43 of spacer 10 have been described as being tapered, such surfaces may alternatively lack a taper. Stated differently, it is contemplated that lead-in surfaces 33, 43 may be rounded, squared-off, pointed, or the like, as opposed to being tapered in the manner described.

Anterior extension 50 of joint spacer 10 may also be modified from the preceding embodiments disclosed. For example, anterior extension 50 may not be circular in cross-section, and rather may be triangular, square, and/or hexagonal in shape. Further, removal geometry 83 of IR instrument 80 may be modified to correspond with the changed shape of anterior extension 50. Likewise, in still other embodiments, aperture 56 in anterior extension 50 may not be square shaped, and may be of any shape, including circular, triangular, hexagonal, or the like. In these embodiments, insertion geometry 81 of IR instrument 80 may also be modified to correspond to the changed shape of aperture 56.

Further, in one embodiment, it is contemplated that the interface between insertion geometry 81 and aperture 56 on anterior extension 50 may not be one of compression. In other words, the dimensions of insertion geometry 81 and aperture 56 may be such that insertion geometry 81 is freely inserted and removed from aperture 56, without compression resulting therebetween. It is also contemplated that anterior extension 50 may be omitted altogether, and spacer 10 may be inserted by hand or via alternate IR instruments.

Although not shown in the figures, it is also contemplated that each of distractors 100, 200 may include removable handles, as opposed to the integral handles 110, 210 depicted. In one embodiment, such removable handles may be inserted within a particularly configured aperture in each of arms 120, 220, and may be removed from arms 120, 220 after such arms 120, 220 have been distracted. As such, handles 110, 210 of distractors 100, 200 need not interfere with surgical preparation of bone during a knee arthroplasty (or other) procedure.

While the invention(s) has been described herein in connection with knee arthroplasty surgery (e.g., a UKR), it is envisioned that the invention(s) may be used for any articulating joint within the body, including, but not limited to, joints in the hip, shoulder, knee, hand, wrist, ankle, or spine. Regarding spinal applications, the invention(s) may be applied by insertion between vertebral body segments in the cervical, thoracic, and/or lumbar regions. The shape and geometry of various portions of spacer 10, of distractors 100, 200, 300, 400, and of inflatable member 500 and/or shim 600 may be modified to accommodate these other joints.

Although the invention(s) herein has been described with reference to particular embodiments, it is to be understood that these embodiments are merely illustrative of the principles and applications of the present invention(s). It is therefore to be understood that numerous modifications may be made to the illustrative embodiments and that other arrangements may be devised without departing from the spirit and scope of the present invention(s) as defined by the appended claims.

It will also be appreciated that the various dependent claims and the features set forth therein can be combined in different ways than presented in the initial claims. It will also be appreciated that the features described in connection with individual embodiments may be shared with others of the described embodiments.

1. A joint spacer comprising:
a body having first, second, and third extensions projecting therefrom, the second and third extensions being spaced apart from one another, each extension having a bone contacting surface,
wherein the first extension lies in a plane extending between the spaced apart second and third extensions and vertically above such extensions, the first extension including a curved portion.

2. The joint spacer of claim 1, wherein the curved portion of the first extension includes a first curved section and a second curved section, the first section curving in an opposite direction to the second section.

3. The joint spacer of claim 2, wherein the second curved section is configured to receive a portion of an intercondylar notch of a femur, and a flat portion between the first curved section and the second curved section is configured to prevent over insertion of the spacer past the intercondylar notch.

4. The joint spacer of claim 1, wherein the second and third extensions project in the same general direction as the first extension, and each extension terminates in a tapered lead-in surface.

5. The joint spacer of claim 1, wherein the bone contacting surfaces of the first, second, and third extensions include serrations.

6. The joint spacer of claim 1, further comprising a fourth extension projecting from the body of the spacer in a direction opposite to the first, second, and third extensions, the fourth extension including a first section having a first diameter, and a second section having a second diameter, the second diameter being greater than the first diameter.

7. The joint spacer of claim 6, wherein the fourth extension includes a bore extending at least partially therethrough.

8. A joint stabilization system comprising:
a joint spacer including:
a body having first, second, and third extensions projecting therefrom, the second and third extensions being spaced apart from one another,
wherein the first extension lies in a plane extending between the spaced apart second and third extensions; and
a combination insertion-removal instrument including:
an insertion member for inserting the spacer between portions of contiguous bone segments; and
a removal member for removing the spacer from between the portions of contiguous bone segments,
wherein the insertion member and the removal member are connected together through an elongate handle.

9. The joint stabilization system of claim 8, wherein the joint spacer further comprises a fourth extension projecting from the body of the spacer in a direction opposite to the first, second, and third extensions.

10. The joint spacer of claim 9, wherein the fourth extension includes a first section having a first diameter, and a second section having a second diameter, the second diameter being greater than the first diameter.

11. The joint stabilization system of claim 9, wherein the insertion member is insertable within a portion of the fourth extension of the spacer.

12. The joint stabilization system of claim 11, wherein the insertion member includes a post configured for insertion within a bore formed in the fourth extension of the spacer, and insertion of the post within the bore secures the insertion-removal instrument to the spacer.

13. The joint stabilization system of claim 10, wherein the removal member is configured to engage the first section of the fourth extension.

14. The joint stabilization system of claim 13, wherein the removal member comprises an open portion that at least partially surrounds the first diameter of the first section upon engaging the removal member with the fourth extension.

15. The joint stabilization system of claim 14, wherein the removal member comprises two tines and a curved portion extending between the tines.

16. A method for stabilizing a knee joint, comprising the steps of:
inserting a spacer having a body between the knee joint, such that a first extension projecting from the body is disposed between the anterior and posterior cruciate ligaments, and second and third extensions projecting from the body are disposed on adjacent sides of such ligaments.

17. The method for stabilizing a knee joint of claim 16, further comprising the step of resecting a surface of the knee joint while the spacer is inserted within the joint.

18. The method for stabilizing a knee joint of claim 16, further comprising the step of removing the spacer from the knee joint.

19. The method for stabilizing a knee joint of claim 18, wherein the inserting and removing steps include engaging a portion of an insertion-removal tool with a fourth extension projecting from the spacer, the fourth extension being oriented in a direction opposite to the first, second, and third extensions.

20. The method for stabilizing a knee joint of claim 19, wherein, during the inserting step, the portion of the insertion-removal tool is a post, which is insertable within a bore formed in the fourth extension; and
during the removing step, the portion of the insertion-removal tool comprises two tines and a curved portion extending between the tines.

21. The method for stabilizing a knee joint of claim 16, wherein the first extension includes a curved portion adapted to mate with an intercondylar notch of a knee joint.

22. The method for stabilizing a knee joint of claim 16, further comprising the step of contacting a portion of the first extension with the intercondylar notch or a portion of a knee adjacent the intercondylar notch to prevent over insertion of the spacer.
